(19) United States
(12) Patent Application Publication
(10) Pub. No.: US 2018/0140455 A1
(43) Pub. Date: May 24, 2018

(54) ADJUSTABLE CERVICAL COLLAR

(71) Applicant: HUNTEX CORPORATION, Taipei (TW)

(72) Inventor: Chia-Chang CHAO, Taipei (TW)

(21) Appl. No.: 15/358,727

(22) Filed: Nov. 22, 2016

(51) Int. Cl. A61F 5/055; F16H 21/44; F16H 19/00; F16H 37/12

(52) U.S. Cl. CPC A61F 5/055; F16H 37/12; F16H 19/001; F16H 21/44

(57) ABSTRACT

An adjustable cervical collar includes a lower support member, an upper support member and two adjusting assemblies. The lower support member includes two axial connecting portions and two installation portions. The upper support member includes two assembly portions respectively pivotally connected to the axial connecting portions and a mandible support portion away from the two assembly portions. The adjusting assemblies are respectively installed at the installation portions, and each includes a first gear, a second gear engaged with the first gear, and a linking member. When the adjustable cervical collar is put to application, the first gear may drive a second section extended from the linking member to push the upper support member upwards through the second gear, thereby causing the mandible support portion to come into contact with and steadily support a mandible of a patient as the upper support member rotates.

### FIELD OF THE INVENTION

The present invention relates to an adjustable cervical collar, and particularly to an adjustable cervical collar that can be easily adjusted by a user.

### BACKGROUND OF THE INVENTION

When a human body receives a significant impact force from an accident (e.g., a car accident, falling off from a building, or falling), bones of various parts may become dislocated or even fractured. In order to prevent these damages on the human body in the above event from aggravating or to thoroughly protected treated areas, a protective gear that limits movement is frequently placed to surround an injured position. In a situation of an injured neck, medical professionals usually place a cervical collar around the neck to limit a moving angle of the neck.

For example, the U. S. Patent Publication No. 2016000597A1 discloses a universally adjustable cervical collar including a base and rearward extending wings. The base includes a front and a chin support. Two sides of the front include respective tracks and respective adjustable latch mechanisms disposed in the respective tracks. In an application of the universally adjustable cervical collar, the vertical position of the chin support is adjusted by changing the positions of the adjustable latch mechanisms in the tracks to adapt to patients with difference neck lengths.

Further, the U. S. Patent Publication No. 2007270728A1 discloses an adjustable neck brace including a cover panel, a height adjusting plate, a support portion and two adjusting devices. Each adjusting device includes a casing installed on the cover panel, an adjusting knob disposed on a surface of the casing, and a connecting portion disposed in the casing and fixed to the height adjusting plate. An adjusting gear of the adjusting knob is engaged at a plurality of protrusions of the connecting portion. By rotating the adjusting knob, the adjustable neck brace may adjust the protrusions to move upwards, so as to adjust the height position of the height adjusting plate to adapt to patients with different neck lengths.

However, it is observed from the two above disclosures that, the adjustable latch mechanism and the chin support in the U. S. Patent Publication No. 2016000597A1 have a direct force receiving relationship. Thus, when the chin support is in contact with the chin, the user cannot easily push the chin support up easily, such that the chin support may fail to steadily support the chin. Further, in the adjustable neck brace in the U. S. Patent Publication No. 2007270728A1, the height adjusting plate and the connecting portion similarly have a direct force receiving relationship. Thus, when the height adjusting plate is in contact with the chin, a user cannot easily push the height adjusting plate upwards, such that the height adjusting plate may fail to steadily support the chin.

Therefore, there is a need for an adjustable cervical collar that is capable of steadily supporting the chin.

### SUMMARY OF THE INVENTION

It is a primary object of the present invention to solve the issue of not being able to steadily support the mandible to due difficulties in applying a force during applications of conventional structures.

To achieve the above object, the present invention provides an adjustable cervical collar including a lower support member, an upper support member and two adjusting assemblies. The lower support member includes two axial connecting portions respectively disposed at two opposite sides of the lower support member, and to two installation portions disposed at the lower support member and respectively away from the two axial connecting portions. Each of the installation portions includes a first axis, an arc track regarding the first axis as a center of circle, and a second axis located between the first axis and the arc track. The upper support member includes two assembly portions disposed respectively at two opposite sides of the upper support member and respectively pivotally connected to the axial connecting portions, a mandible support portion disposed at the upper support portion and away from the two assembly portions, and two bridge portions located between the respective assembly portions and the mandible support portion and respectively aligned with the arc tracks. The two adjusting assemblies are respectively installed at the installation portions. Each of the adjusting assemblies includes a first gear disposed correspondingly to one of the first axes, a second gear disposed correspondingly to one of the second axes and engaged with one of the first gears, and a linking member assembled with one of the first gears. Each of the second gears includes an eccentric column. Each of the linking member includes a first section assembled with one of the first gears, a second section disposed correspondingly to one of the arc tracks and assembled with one of the bridge portions, and a connecting section located between the first section and the second section. Each of the connecting sections includes a hollow region for inserting the eccentric column. The second gear is driven by the first gear to drive and move the linking member through the eccentric column, such that the upper support member rotates and moves relative to the lower support member by regarding the two axial connecting portions as an axis.

In one embodiment, the bridge portions are respectively strip-like tracks respectively aligned with the arc tracks. Further, each of the linking member includes a protrusion, which is disposed at one of the second sections and extends into one of the arc tracks and one of the strip-like tracks to move the upper support member.

In one embodiment, the adjustable cervical collar further includes two positioning assemblies respectively adjacent to the first axes and operable to secure a position of the upper support member. Further, the lower support member includes two installation grooves respectively protruding from the two first axes towards opposite inner sides. Each of the positioning assemblies includes a third gear connected to an axial position of one of the first gears and located in one of the installation grooves, a step limiting member fixed in one of the installation grooves and engaged with one of the third gears, and a C-shaped limiting member attached to an inner wall of one of the installation grooves and rotatable to extend to an outer side of the step limiting member to cause that step limiting member to limit one of the third gears from rotating. The lower support member further includes two notches respectively provided at sidewalls of the installation grooves. Each of the C-shaped limiting member includes an operation plate, which extends out of one of the notches and adapted to receive a press to rotate one of the C-shaped limiting members.

In one embodiment, the adjustable cervical collar further includes two rotatable knobs respectively connected to the axial positions of the first gears to drive the respective first gears to rotate. The adjustable cervical collar further includes two cover plates respectively installed at the installation portions. Each of the cover plates includes an opening that reveals one of the rotatable knobs, and a plurality of through holes disposed along one of the arc tracks and observable to learn a position of one of the connecting sections.

In one embodiment, the lower support member includes a front plate, two lateral plates connected to two opposite sides of the front plate and for disposing the respective axial connecting portions and the respective installation portions, two elastic band bodies respectively connected to the lateral plates, a rear plate connected to the two elastic band bodies, and an accommodating space formed at inner sides of the front plate, the two lateral plates and the rear plate. The upper support member further includes two connecting plates for disposing the respective assembly portions and the respective bridge portions. The mandible support portion is pivotally connected between the two connecting plates and away from the two assembly portions.

With the above implementation of the present invention, the present invention provides following features as opposed to the prior art. In the present invention, the linking members are driven by the first gears through the second gears to push the upper support member, such that forces of the eccentric columns of the respective second gears for pushing the respective linking members to rotate are reduced using the torques that the respective second sections and the respective connecting sections produce. Thus, a user is allowed to rotate the first gears with minimal effort to adjust the position of the upper support member to steadily support the mandible, thereby solving the issue of not being able to steadily support the mandible to due difficulties in applying a force during applications of conventional structures.

### BRIEF DESCRIPTION OF THE DRAWINGS

FIG. 1 is a perspective view of the present invention;

FIG. 2 is a first exploded view of the present invention;

FIG. 3 is a second exploded view of the present invention;

FIG. 4 is a side view of the present invention;

FIG. 5A is a first partial side view of the present invention in operation;

FIG. 5B is a second partial side view of the present invention in operation;

FIG. 6A is a first partial side view of the present invention; and

FIG. 6B is a second partial side view of the present invention.

### DETAILED DESCRIPTION OF THE PREFERRED EMBODIMENTS

Details and technical contents of the present invention are given with the accompanying drawings below.

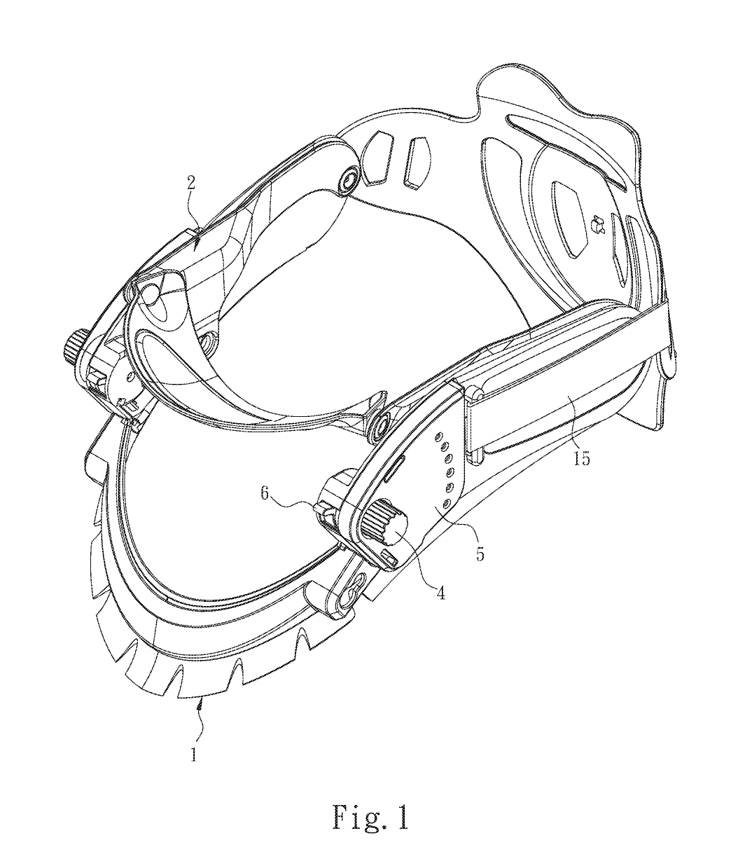

Referring to FIG. 1 to FIG. 3, the present invention provides an adjustable cervical collar including a lower support member 1, an upper support member 2, and two adjusting assemblies 3. The lower support member 1 includes two axial connecting portions 11 and two installation portions 12. The two axial connecting portions 11 are respectively disposed at two opposite sides of the lower support member 1. The two installation portions 12 are disposed on a surface of the lower support member 1 and away from the two axial connecting portions 11. Each of the installation portions 12 includes a first axis 121, an arc track 122 and a second axis 123. Each of the arc tracks 122 appears as an arc pattern by regarding the corresponding first axis 121 as a center of circle. Each of the second axes 123 is located between the corresponding first axis 121 and the corresponding arc track 122. In one embodiment, the lower support member 1 is formed by a front plate 13, two lateral plates 14, two elastic band bodies 15 and a rear plate 16. Two opposite sides of the front plate 13 are respectively connected to the two lateral plates 14. The two lateral plates 14 are parallel and are for disposing the respective axial connecting portions 11 and the respective installation portions 12. Two opposite sides of the rear plate 16 are respectively connected to the two lateral plates 14 respectively through the two elastic band bodies 15. An accommodating space 10 is formed at inner sides of the front plate 13, the lateral plates 14 and the rear plate 16. Observing the appearance of the formed structure of the front plate 13, the lateral plates 14 and the rear plate 16, the formed structure of the front plate 13, the lateral plates 14 and the rear plate 16 adapts an upper chest, shoulders and a rear neck of the human body. More specifically, the shape of the inner side of the front plate 13 conforms with the upper chest, the shape of lower sides of the two lateral plates 14 conform with the shoulders, and the inner side of the rear plate 16 conforms with the rear neck. As such, the lower support member 1 may be steadily placed and accommodated around the lower neck of the human body.

The upper support member 2 includes two assembly portions 21, a mandible support portion 22 and two bridge portions 23. The two assembly portions 21 are disposed at two opposite sides of the upper support member 2 and are pivotally connected to the axial connecting portions 11, respectively. The mandible support portion 22 is disposed at a position at the upper support member 2 and away from the two assembly portions 21. The bridge portions 23 are located between the respective assembly portions 21 and the mandible support portion 22, and are respectively aligned with the arc tracks 122. More specifically, the two assembly portions 21 and the two axial connecting portions 11, through mutually assembled axial columns and axial openings, may cause the upper support member 2 to rotate and move relative to the lower support member 1 by regarding a straight line formed by connecting the two axial connecting portions 11 as an axis. In one embodiment, the upper support member 2 includes two connecting plates 24, which are for to disposing the respective assembly portions 21 and the respective bridge portions 23. The mandible support portion 22 is pivotally connected at positions at the two connecting plates 24 away from the two assembly portions 21. The appearance of the mandible support portion 22 conforms with a mandible of the human body, such that the mandible support portion 22 may be closely placed at a surface of the mandible after rotating between the two connecting plates 24.

Technical features of the lower support member 1 and the upper support member 2 can be clearly understood from the above description. Structural details of the two adjusting assemblies 3 and assembly relationships of the two adjusting assemblies 3 with the lower support member 1 and the upper support member 2 are illustrated below. More specifically, the two adjusting assemblies 3 are correspondingly disposed at the installation portions 12, respectively. Each of the adjusting assemblies 3 includes a first gear 31, a second gear 32 and a linking member 33. The first gears 31 are installed correspondingly at the first axes 121, respectively. The second gears 32 are correspondingly installed at the second axes 123, respectively, and are engaged with the respective first gears 31. The linking members 33 are assembled with the first gears 31, respectively. Further, each of the first gears 31 and one of the linking members 33 are located at the same axis and are capable of rotating independently. Each of the second gears 32 includes an eccentric column 321 (i.e., a column departing from an axial position of the second gear 32) of the on its surface. Each of the linking members 33 includes a first section 331, a second section 332 and a connecting section 333. The first sections 331 are assembled with the first gears 31, respectively. The second sections 332 are disposed correspondingly to the arc tracks 122, respectively, and are assembled with the bridge portions 23, respectively. The connecting sections 333 respectively are located between the respective first sections 331 and the respective second sections 332, and each includes a hollow region 334 for inserting one of the eccentric columns 321. In one embodiment, each of the bridge portions 23 is a strip-like track aligned with one of the arc tracks 122. Each of the linking members 33 includes a protrusion 335, which is disposed at one of the second sections 332 and extends into one of the arc tracks 122 and one of the strip-like tracks. Accordingly, the two linking members 33 may push the upper support member 2 upwards through the respective protrusions 335 when rotated.

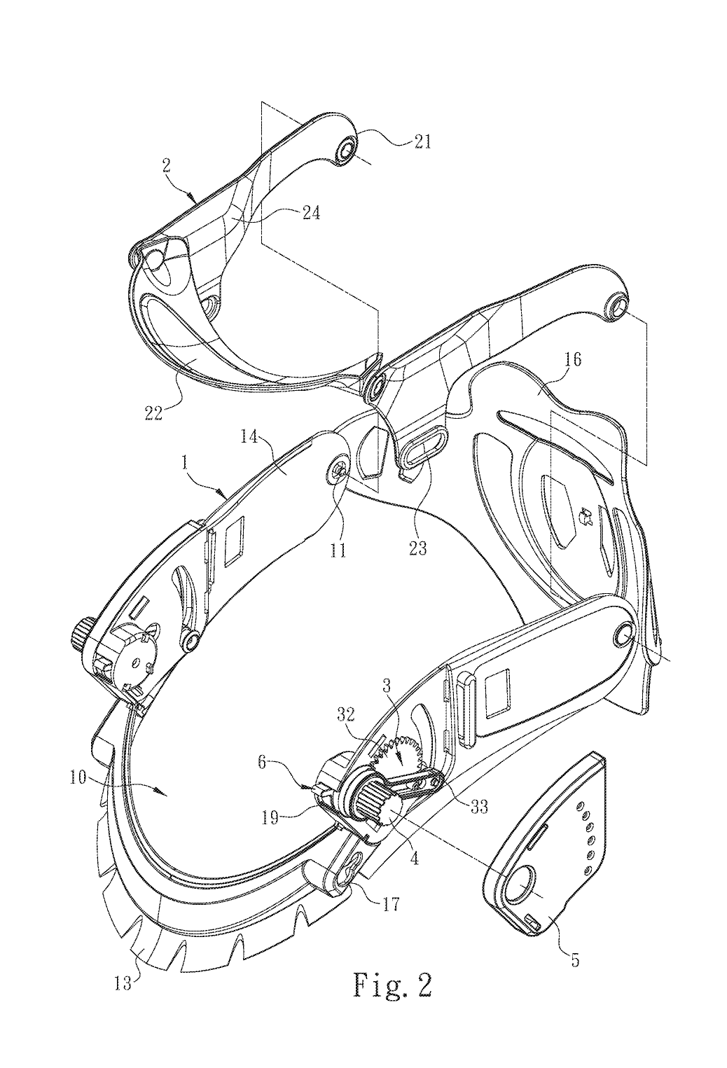

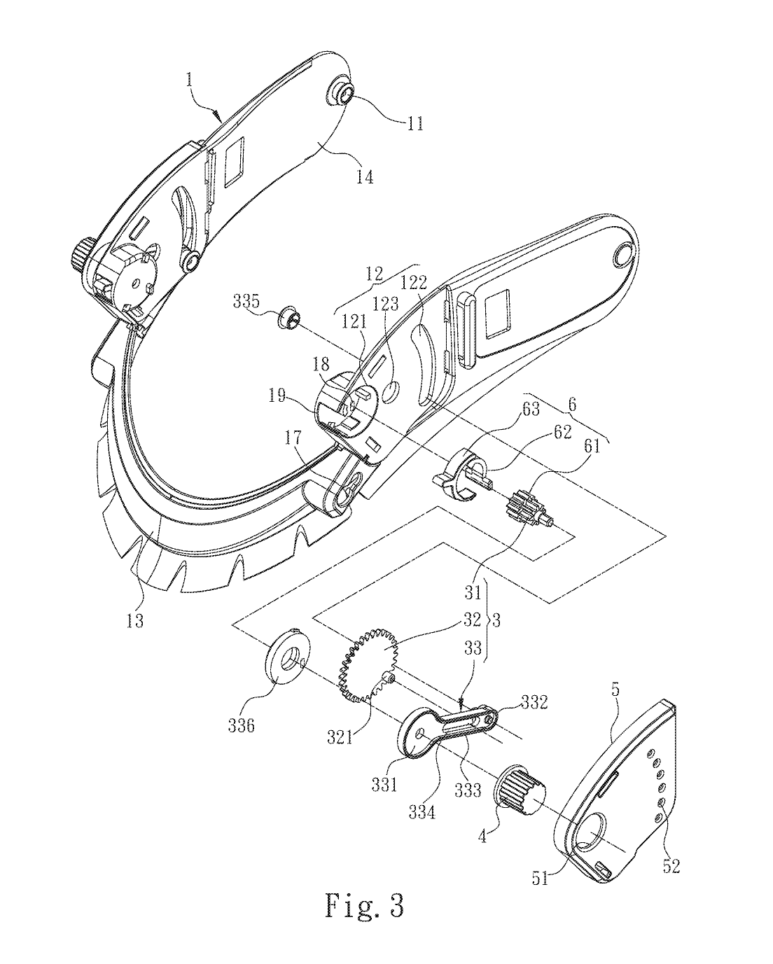

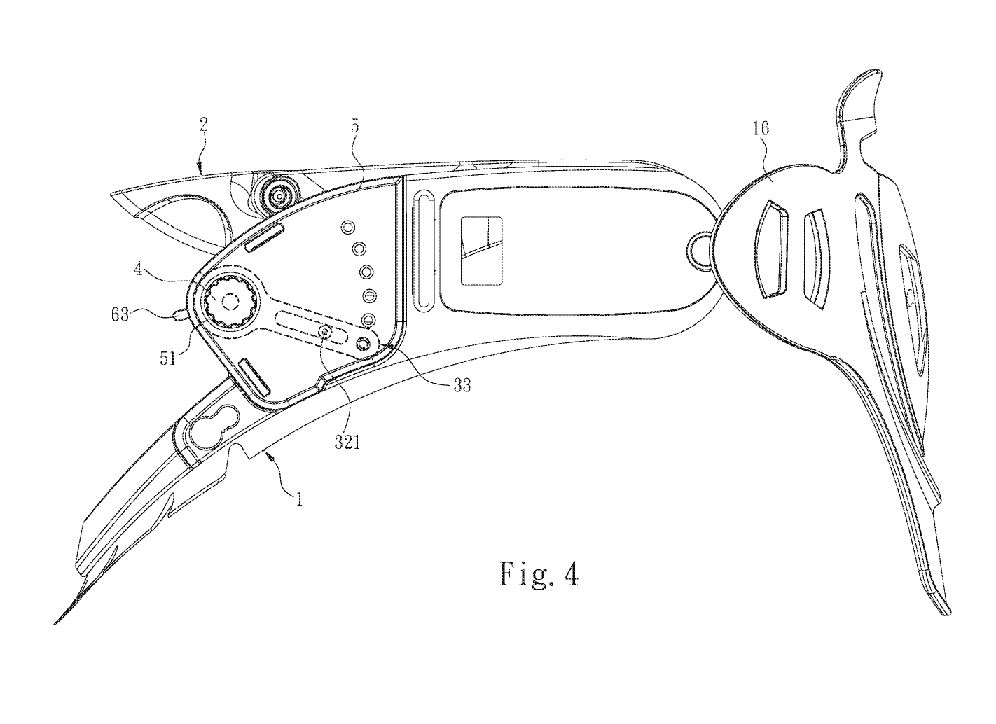

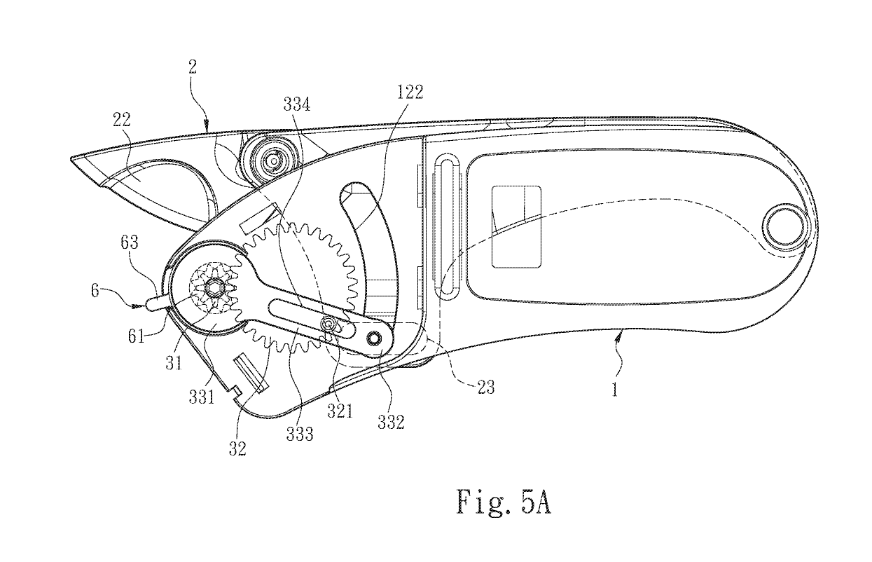

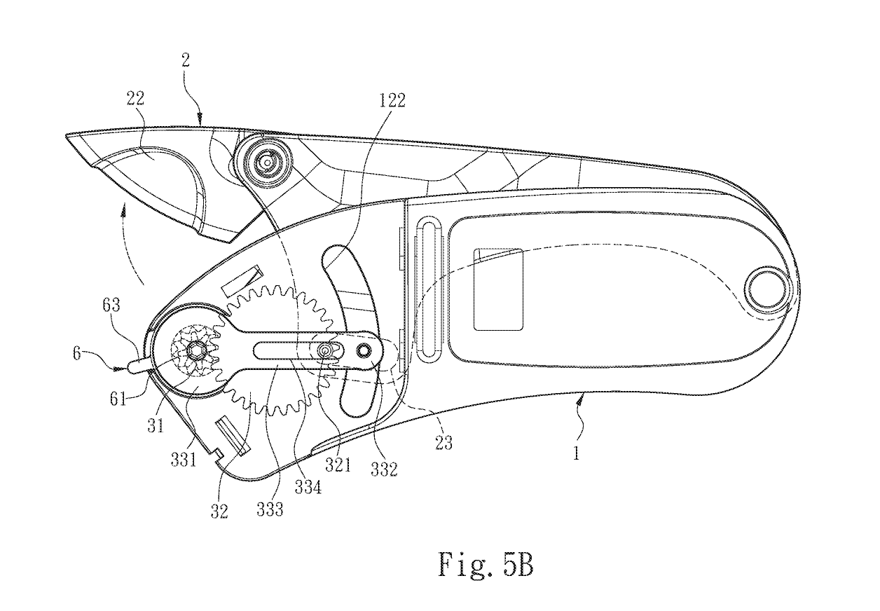

The structural details of the lower support member 1, the upper support member 2 and the two adjusting assemblies 3 are as described above. In the following, an application process of the adjustable cervical collar of the present invention is described in detail. It should be noted that, as application processes of the two adjusting assemblies 3 are identical, the operation relationship of only one of the adjusting assemblies 3 with the lower support member 1 and the upper support member 2 is illustrated below. When the present invention is put to application, as shown in FIG. 2 to FIG. 5B, the lower support member 1 is first accommodated around the neck of a patient, to cause a lower side of the lower support member 1 to be closely placed at the upper chest, shoulders, upper back and rear neck of the patient. In one embodiment, the lower support member 1 includes two assembly holes 17 (as shown FIG. 3) respectively disposed at the two opposite sides of the front plate 13. Each of the two assembly holes 17 may be installed by a support rod (not shown) to securely connect to a waist support member (not shown). Next, the first gear 31 rotates to drive the second gear 32 to rotate, so as to cause the eccentric column 321 to rotate round the axis of the second gear 32 and to drive the connecting section 333 to rotate by regarding the first axis 121 as an axis in the hollow region 334. In one embodiment, the adjustable cervical collar of the present invention further includes two rotatable knobs 4 (as shown in FIG. 3) connected to the axial positions of the two first gears 31, respectively. The rotatable knob 4 is rotatable to drive the first gear 31 to drive the second gear 32 and the linking member 33 to rotate. During the process in which the connecting section 333 rotates by regarding the first axis 121 as an axis, the second section 332 drives the bridge portion 23 to move upwards or downwards in the arc track 122 along with the rotation of the connecting section 333. Thus, the upper support member 2 is allowed to rotate and move relative to the lower support member 1 by regarding the two axial connecting portions 11 as an axis, so as to further cause the mandible support portion 22 to rotate with the upper support member 2 to support the mandible of the patient, as shown in FIG. 5A and FIG. 5B. In one embodiment, the adjustable cervical collar further includes two cover plates 5 installed at the installation portions 12, respectively (as shown in FIG. 2 and FIG. 4). Each of the cover plates 5 includes an opening 51 and a plurality of through holes 52. The opening 51 reveals the rotatable knob 4, and the through holes 52 are disposed along the arc track 122 for the patient to observe and learn the position of the connecting section 333. In conclusion, because the first gear 31 drives the second section 332 extended from the linking member 33 through the second gear 32 to push the upper support member 2, through a torque produced by the second section 332 and the connecting section 333, the eccentric column 321 of the second gear 32 is able to reduce a thrust for driving and rotating the linking member 33. As such, the patient is allowed to rotate the first gear 31 with minimal effort to adjust the position of the upper support member 2 to steadily support the mandible, thereby solving the issue of not being able to steadily support the mandible to due difficulties in applying a force during applications of conventional structures.

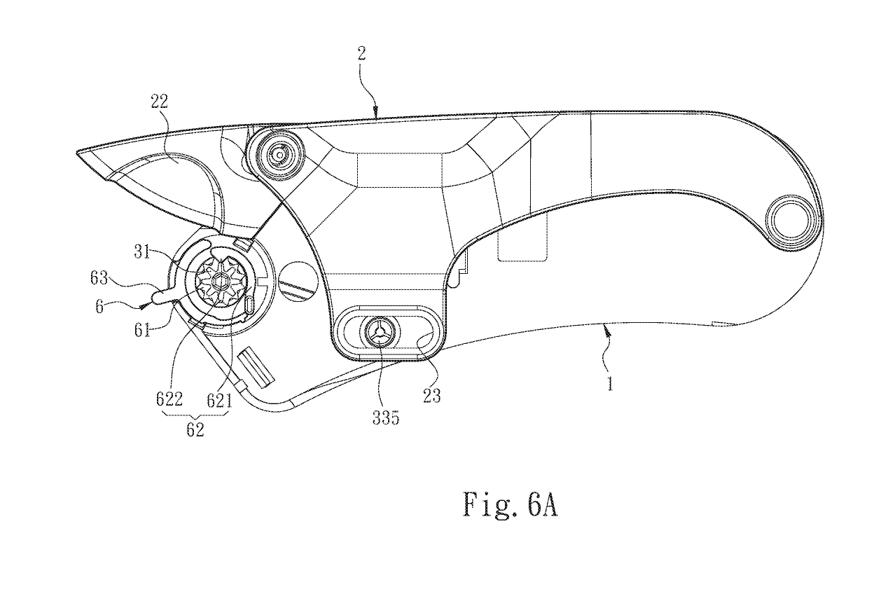

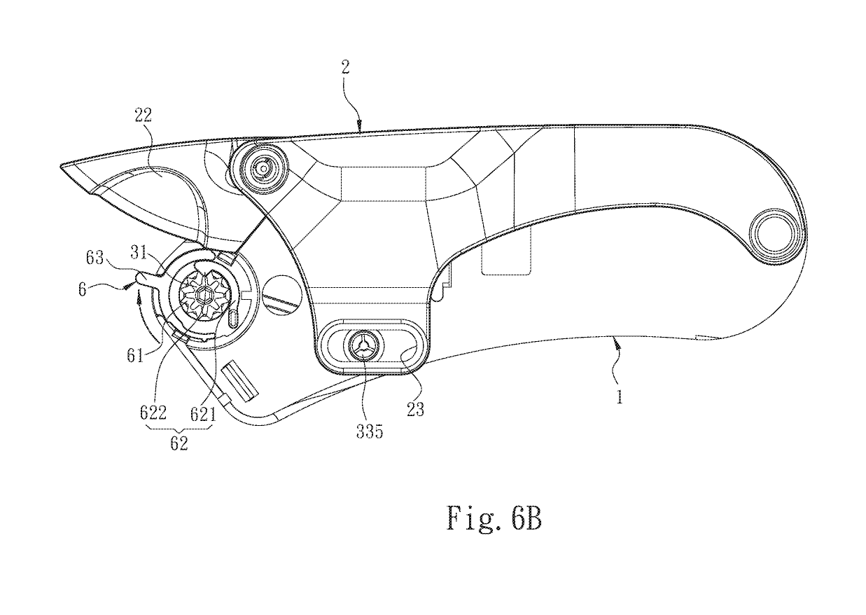

To reinforce the positioning strength of the upper support member 2 after the position of the upper support member 2 is adjusted, the adjustable cervical collar may further include two positioning assemblies 6. The two positioning assemblies 6 are located adjacent to the first axes 121, respectively, and are operable by the patient to fix the position of the upper support member 2, as shown in FIG. 5A to FIG. 6B. In one embodiment, the lower support member 1 further includes two installation grooves 18, and each of the positioning assemblies 6 includes a third gear 61, a step limiting member 62 and a C-shaped limiting member 63. The two installation grooves 18 protrude from the two first axes 121 towards opposite inner sides. The two third gears 61 are respectively connected to the axial positions of the first gears 31, and are respectively located in the installation grooves 18. The two step limiting members 62 are respectively fixed in the installation grooves 18, and are respectively engaged with the third gears 61. Each of the two C-shaped limiting members 63 may be formed by an arc rod 621 and a protrusion 622. The protrusion 622 is disposed at one end of the arc rod 621, and is engaged with the corresponding third gear 61. Further, each of the adjusting assemblies 3 may further include a spacer plate 336. The two spacer plates 336 are respectively installed in the installation grooves 18 to respectively limit the third gears 61 in the respective installation grooves 18. In the application process of this embodiment, the protrusion 622 of the step limiting member 62 may be abutted outwards by the corresponding third gear 61 as the third gear 61 rotates, and then restore inwards to fasten the third gear 61. The C-shaped limiting member 63 may be rotated by the patient to extend between an outer side of the corresponding step limiting member 62 and the installation groove 18 (as shown in FIG. 6B). Thus, the third gear 61 is limited from rotating through the step limiting member 62, such that the first gear 31, the second gear 32 and the linking member 33 are positioned as the third gear 61 is secured to further cause the upper support member 2 to be positioned at a fixed position. Further, referring to FIG. 3, the lower support member 1 may include two notches 19, and each of the C-shaped limiting members 63 may include an operation plate 631. The two notches 19 are respectively provided at sidewalls of the installation grooves 18, and the two operation plates 631 respectively extend to outer sides of the notches 19. Further, the patient may press the operation plate 631 to rotate the C-shaped limiting member 63 to limit the third gear 61 from rotating.

What is claimed is:

1. An adjustable cervical collar, comprising:
a lower support member, comprising two axial connecting portions disposed respectively at two opposite sides of the lower support member and two installation portions disposed at the lower support members and respectively away from the two axial connecting portions, each of the installation portions comprising a first axis, an arc track regarding the first axis as a center of circle and a second axis located between the first axis and the arc track;
an upper support member, comprising two assembly portions disposed respectively at two opposite sides of the upper support member and respectively pivotally connected with the axial connecting portions, a mandible support portion disposed at the upper support member and away from the two assembly portions, and two bridge portions respectively located between the respective assembly portions and the mandible support portion and respectively aligned with the arc tracks; and
two adjusting assemblies, respectively installed at the installation portions, each of the adjusting assemblies comprising a first gear disposed correspondingly to one of the first axes, a second gear disposed correspondingly to one of the second axes and engaged with one of the first gears, and a linking member assembled with one of the first gears respectively, each of the second gears comprising an eccentric column, each of the linking members comprising a first section assembled with one of the first gears, a second section disposed correspondingly to one of the arc tracks and assembled with one of the bridge portions, and a connecting section located between the first section and the second section, each of the connecting sections comprising a hollow region for inserting one of the eccentric columns, the second gears being driven by the respective first gears to drive the respective linking members to move to cause the upper support member to rotate and move relative to the lower support member by regarding the two axial connecting portions as an axis.

2. The adjustable cervical collar of claim 1, wherein each of the bridge portions is a strip-like track aligned with one of the arc tracks.

3. The adjustable cervical collar of claim 2, wherein each of the linking members further comprises a protrusion, which is disposed at one of the second sections and extends into one of the arc tracks and one of the strip-like tracks to be able to push the upper support member upwards.

4. The adjustable cervical collar of claim 1, further comprising two positioning assemblies, which are respectively located adjacent to the first axes and are operable to fix a position of the upper support member.

5. The adjustable cervical collar of claim 4, wherein the lower support member comprises two installation grooves protruding from the two first axes towards opposite inner sides, each of the positioning assemblies comprises a third gear connected to an axial position of one of the first gears and located in one of the installation grooves, a step limiting member fixed in one of the installation grooves and engaged with one of the third gears, and a C-shaped limiting member attached to an inner wall of the one of the installation grooves and rotatable to extend to an outer side of one of the step limiting members to cause the step limiting member to limit one of the third gears from rotating.

6. The adjustable cervical collar of claim 5, wherein the lower support member further comprises two notches respectively provided at sidewalls of the installation grooves, and each of the C-shaped limiting members comprises an operation plate extending out of one of the notches and adapted to be pressed to rotate one of the C-shaped limiting member.

7. The adjustable cervical collar of claim 1, further comprising two rotatable knobs respectively connected to axial positions of the two first gears to drive the respective first gears to rotate.

8. The adjustable cervical collar of claim 7, further comprising two cover plates respectively installed at the installation portions, and each of the two cover plates comprises an opening that reveals one of the rotatable knobs and a plurality of through holes disposed along one of the arc tracks to be observed and learn a position of one of the connecting sections.

9. The adjustable cervical collar of claim 1, wherein the lower support member further comprises a front plate, two lateral plates connected to two opposite sides of the front plate and respectively for disposing the axial connecting portions and the installation portions, two elastic band bodies respectively connected to the lateral plates, a rear plate connected to the two elastic band bodies, and an accommodating space formed at inner sides of the front plate, the two lateral plates and the rear plate, the upper support member further comprises two connecting plates for respectively disposing the respective assembly portions and the respective bridge portions, and the mandible support portion is pivotally connected between the two connecting plates and away from the two assembly portions.
